(19) United States
(12) Patent Application Publication
(10) Pub. No.: US 2021/0275403 A1
(43) Pub. Date: Sep. 9, 2021

(54) FLUORIDE RELEASING COMPOSITION AND METHOD OF USE

(71) Applicant: 3M INNOVATIVE PROPERTIES COMPANY, St. Paul, MN (US)

(72) Inventors: Yizhong Wang, Woodbury, MN (US); Joel D. Oxman, Minneapolis, MN (US)

(21) Appl. No.: 16/475,404

(22) PCT Filed: Feb. 5, 2018
(86) PCT No.: PCT/US2018/016805

§ 371 Date: Jul. 2, 2019

Related U.S. Application Data
(60) Provisional application No. 62/457,235, filed on Feb. 10, 2017.

(51) Int. Cl. A61K 6/20; A61K 6/70; A61K 6/60; A61C 19/06

(52) U.S. Cl. CPC A61K 6/20; A61C 19/063; A61K 6/60; A61K 6/70

(57) ABSTRACT

Fluoride releasing compositions comprising water, ethanol, partially neutralized hydrogenated acid rosin, and fluoride releasing agent. The fluoride releasing compositions are often in the form of dental varnishes. Methods of using the same, such as by application to one or more teeth.

### CROSS REFERENCE TO RELATED APPLICATIONS

This application claims the benefit of U.S. Provisional Patent Application No. 62/457,235, filed Feb. 10, 2017, the disclosure of which is incorporated by reference in its entirety herein.

### BACKGROUND

Dental compositions, such as dental varnish compositions with fluoride sources, have been described, for example, in U.S. Pat. No. 7,335,691. Such compositions typically require a relatively large amount of volatile liquids, such as one or more of ethanol, hydrocarbons, and the like, to dissolve or disperse all of the components.

### SUMMARY

A fluoride releasing composition acceptable for oral use can comprise 50% to 70% by weight of a hydrogenated rosin acid, wherein greater than 17% and less than 85% of the hydrogenated rosin acid is in the form of neutralized hydrogenated rosin acid; 5% to 12% by weight of water; 12% to 20% by weight of ethanol; and one or more fluoride sources comprising fluoride. The fluoride releasing composition has a consistency of at least 2 mm. A method can comprise applying the fluoride releasing composition to the oral cavity, such as to one or more teeth.

### DETAILED DESCRIPTION

Throughout this disclosure, singular forms such as “a,” “an,” and “the” are often used for convenience; however, it should be understood that the singular forms are meant to include the plural unless the singular alone is explicitly specified or is clearly indicated by the context. When the singular alone is called for, the term “one and only one” is typically used.

All percentages (%) in this disclosure are by weight, unless specified otherwise.

Dental compositions, such as dental varnish compositions with fluoride, can contain rosins, which can assist in setting the composition in the oral cavity. As used in this disclosure, a “set” composition or a composition “setting” do not necessarily require any chemical reaction, such as a polymerization or cross-linking reaction. Rather, setting refers to the process by which the composition forms a film, typically on the teeth, for example after application to the oral cavity or after contact with saliva. A composition that has formed such a film is herein said to have “set.” Other grammatical forms of the words “set” and “setting” are used in the same context in this disclosure.

In this disclosure, a hydrogenated acid rosin is most commonly used in the fluoride releasing composition. Hydrogenated acid rosins typically have a hydrophobic part and a carboxylic acid functional group. Dispersing, which can include one or both of dissolving and forming a dispersion with sufficient stability for oral or dental use, such hydrogenated acid rosins in aqueous formulations can be difficult. Large amounts of ethanol or other organic solvent can be used, however, in such cases setting the composition may require a drying step or replacement of solvent with saliva. Also, large amounts of ethanol may lead to patient discomfort.

This disclosure provides fluoride releasing composition that can comprise 50% to 70% by weight of a hydrogenated rosin acid, wherein greater than 17% and less than 85% of the hydrogenated rosin acid is in the form of neutralized hydrogenated rosin acid. The fluoride releasing compositions also include 5% to 12% by weight of water, 12% to 20% by weight of ethanol; and one or more fluoride sources comprising fluoride. The fluoride releasing composition typically has a consistency of at least 24 mm. Such compositions can have a consistency that is sufficiently high to allow them to be applied to the oral cavity, particularly to the teeth in the form of a dental varnish, without being washed away by the saliva before setting, while still having a low enough ethanol content that patient discomfort can be mitigated.

Any suitable hydrogenated acid rosin can be used. Typically, the hydrogenated acid rosin will have a carboxylic acid moiety and a hydrophobic moiety. Suitable hydrogenated acid rosins are known in the art, and are available under trade designations such as Foral AX (Pinova Inc. Brunswick Ga.) and Staybelite (Pinova Inc.).

The hydrogenated acid rosin is generally present between 50% and 70% by weight of the fluoride releasing composition. For example, the hydrogenated acid rosin can be present in at least 50%, at least 55%, at least 60%, or at least 65% by weight of the fluoride releasing composition. The hydrogenated acid rosin can be present in no more than 65%, no more than 60%, or no more than 55% by weight of the fluoride releasing composition.

The hydrogenated acid rosin is partially neutralized, such that some of the carboxylic acid moieties are transformed into carboxylate salts. The degree of neutralization is typically greater than 17% and less than 85%. The degree of neutralization can be greater than 17%, greater than 20%, greater than 25%, greater than 30%, greater than 35%, greater than 40%, greater than 45%, greater than 50%, greater than 55%, greater than 60%, greater than 65%, greater than 70%, greater than 75%, greater than 75%, or greater than 80% by weight of the fluoride releasing composition. The degree of neutralization can be less than 85%, less than 80%, less than 75%, less than 70%, less than 65%, less than 60%, less than 55%, less than 50%, less than 45%, less than 40%, less than 35%, less than 30%, less than 25%, or less than 20% by weight of the fluoride releasing composition. Higher degrees of neutralization can adversely affect the ability of the fluoride releasing composition to set. Lower degrees of neutralization can make it difficult or impossible to disperse the composition without using high quantities of ethanol.

The designated degree of neutralization can be obtained in any suitable manner. Typically, the designated degree of neutralization is obtained by adding a sufficient amount of a base to the formulation. Any suitable base can be used. In most cases, the base is an alkali metal or alkaline earth metal hydroxide. Sodium hydroxide and potassium hydroxide are most common. The amount of base, such as sodium hydroxide or potassium hydroxide, to be added can be readily calculated by a person of skill in the art, will depend on the amount of hydrogenated acid rosin used and the acid number of the hydrogenated acid rosin. The acid number of the hydrogenated acid rosin is in many cases known the person of ordinary skill in the art, but if not it can be readily determined by known methods, such as by titration with potassium hydroxide.

The fluoride releasing composition contains 5% to 12% by weight of water. Typically, the water is added to the fluoride releasing composition in the form of distilled, deionized (DI), or reverse-osmosis (RO) water. The amount of water can be at least 5%, at least 6%, at least 7%, at least 8%, at least 9%, at least 10%, or even at least 11% by weight of the fluoride releasing composition. The amount of water can be no more than 12%, no more than 11%, no more than 10%, no more than 9%, no more than 8%, no more than 7%, or no more than 6% by weight of the fluoride releasing composition.

The fluoride releasing composition contains 12% to 20% by weight of ethanol. This is a smaller amount of ethanol than is found in many previous compositions. The amount of ethanol can, in many cases, improve patient acceptance, reduce patient discomfort, and also not interfere with setting. The ethanol can be present in at least 12%, at least 13%, at least 14%, at least 15%, at least 16%, at least 17%, at least 18%, or at least 19% by weight of the fluoride releasing composition. The ethanol can be no more than 20%, no more than 19%, no more than 18%, no more than 17%, no more than 16%, no more than 15%, no more than 14%, or no more than 13% by weight of the fluoride releasing composition. The ethanol is typically added to the fluoride releasing composition as 200 proof ethanol that is safe for oral use. However, it is also possible to add the ethanol as an ethanol/water mixture, in which case the amount of water in the ethanol/water mixture will be counted towards the total amount of water in the composition.

A fluoride source is also included in the fluoride releasing composition. Any suitable fluoride source can be used. Typically, the fluoride source will be acceptable for oral use. Fluoride sources that can be used include, without limitation, one or more of sodium fluoride, strontium fluoride, bismuth fluoride, stannous fluoride, zinc fluoride, potassium fluoride, calcium fluoride, zirconium fluoride, sodium mono-fluorophosphate, hexafluorosilicic acid, and sodium hexafluorosilicate. Sodium fluoride is most common.

Typically, the fluoride source is included in amounts of 0.1% to 10% by weight of the fluoride releasing composition. In some cases, the fluoride source is present in at least 0.1%, at least 0.5%, at least 1%, at least 2%, at least 3%, at least 4%, at least 5%, at least 6%, at least 7%, at least 8%, or at least 9% by weight of the fluoride releasing composition. The fluoride source can be present in no more than 10%, no more than 9%, no more than 8%, no more than 7%, no more than 6%, no more than 5%, no more than 4%, no more than 3%, no more than 2%, no more than 1%, or no more than 0.5% by weight of the fluoride releasing composition. Particular amounts of source that can be present are 2.2% by weight. 2.6% by weight, and 5% by weight of the fluoride releasing composition.

The fluoride releasing composition will typically have a consistency of at least 24 mm. Consistency can be measured by methods known in the art, such as those described in WO2015/038580. All consistency values reported in this disclosure refer to those made by placing 0.15 g of liquid in the center of a first square glass plate having a side length of 10.16 cm (4 inches), suspending a second square glass plate having a side length of 10.16 cm (4 inches) and a mass of 110 g just above the sample, releasing the second glass plate without applying any downward pressure, allowing the second glass plate to remain in place for 120 seconds after release, removing the second glass plate without disturbing the circle of sample formed on the first glass plate, and then measuring the diameter of the circle of sample formed on the first glass plate in three locations at 120 degree intervals. The three measurements are made to the nearest 0.79 mm ( 1/32 inch), and the average of the three measurements is reported.

The fluoride releasing composition is in most cases free of hydrocarbon solvents. However, even in such cases, trace amounts of hydrocarbon solvent may still be present, particularly when one or more of the components of the composition is added as a solution of dispersion in hydrocarbons.

The fluoride releasing composition is, in most cases, free of alcohol solvents other than ethyl alcohol. However, even in such cases alcohols other than ethyl alcohol may be present in trace amounts, particularly when one or more of the components of the composition is added as a solution of dispersion in alcohols other than ethyl alcohol. Alcohols that are not solvents, such as excipients or active agents that have alcohol functional groups, may still be included.

The fluoride releasing composition can include at least one excipient, and most often includes more than one excipient. Typical excipients that can be used are excipient selected from the group consisting of fillers, flavorings, and, sweeteners.

Any orally acceptable sweetener can be used. Common sweeteners include xylitol, sorbitol, aspartame, saccharin, usually sodium saccharine, and the like. When present, the one or more sweeteners can be used in any suitable amount, most often in an amount sufficient to impart a pleasant sweetness to the composition. The suitable amount is typically from 0.5% to 3% by weight of the fluoride releasing composition.

Any orally acceptable flavoring can be used. Common flavorings include peppermint oil, spearmint oil, cherry flavor, citric acid, orange flavor, vanilla, strawberry flavor, coconut flavor, and bubble gum flavor. When present, flavoring agents are typically used in amounts from 1%-4% by weight of the fluoride releasing composition.

Any orally acceptable filler can be used. Typical fillers include one or more of polymers, such as polyethylene glycol, polypropylene glycol, and copolymers of polyethylene glycol and polypropylene glycol, silica, such as fumed silica, alumina, titania, zinc oxide, and glycerin. When used, the fillers are most commonly present in 0.5% to 15% by weight, although other amounts are also possible.

One or more rosin esters can be included in the composition. Rosin esters are acid rosins that have been modified by an esterification condensation reaction with one or more alcohols. Any orally acceptable rosin acid can be used. Examples of rosin esters include methyl rosin esters, triethylene glycyl (mono-, di-, or tri-condensate) rosin esters, glycerol (mono- or di-condensate) rosin esters, and pentaerythryl rosin esters. When included, rosin esters are typically present from 5% to 10% by weight, such as at least 5%, at least, 6%, at least 7%, at least 8%, or at least 9% by weight. Rosin esters can also be present in no more than 10%, no more than 9%, no more than 8%, no more than 7%, or no more than 6% by weight.

The fluoride releasing composition can also contain one or more additional active agents. When included, the one or more additional active agents usually, but not always, include one or more active agents that are active in the oral cavity against disorders, diseases, or conditions of the teeth, gums, cheeks, tongue, roof of the mouth, and the like.

Examples of the one or more additional active agents that can be employed include one or more whitening agents, anticalculus agents, remineralization agents, stannous sources, antimicrobial agents, antioxidants, saliva stimulating agents, breath freshening agents, antiplaque agents, anti-inflammatory agents, H₂ antagonists, desensitizing agents, nutrients, and proteins. When employed, the one or more additional active agents will be typically be used in amount sufficient to achieve their intended effect.

When employed, the one or more whitening agents can be any suitable whiting agents. The one or more whitening agents can include, for example, a peroxide whitening agent, a non-peroxide whitening agent, or both. Peroxide whitening agents include hydrogen peroxide, peroxide of alkali or alkaline earth metals, such as sodium peroxide, potassium peroxide, lithium peroxide, magnesium peroxide, calcium peroxide, barium peroxide, and the like, glyceryl hydrogen peroxide, alkyl hydrogen peroxide, dialkyl peroxide, peroxy acids or peroxy acid salts, benxoyl peroxide, urea peroxide, and the like. Hydrogen peroxide is most common. Non-peroxide whitening agents include chlorine dioxide, chlorites, and hypochlorites. Chlorites and hyperchlorites are typically in the form of alkali or alkaline earth metal salts, such as salts of lithium, potassium, sodium, magnesium, calcium, or barium. Colorants, titanium dioxide, and hydroxyapatite can also be used.

When employed, the one or more anticalculus agents can be any suitable anticalculus agents. The one or more anticalculus agents can include, for example, phosphates, polyphosphates, such as pyrophosphates, polyaminopropanesulfonic acid, polyolefin sulfonates, polyolefin phosphates, diphosphnates, phosphonoalkane carboxylic acids, and salts thereof, typically alkali metal or ammonium salts.

When employed, the one or more remineralization agents can be any suitable remineralization agents. The one or more remineralization agents can include, for example, materials that release calcium ions, phosphorous-containing ions, or both, such as calcium phosphate (e.g., mono-, di-, and/or tricalcium phosphate), hydroxyapatite, calcium carbonate, and the like.

When employed, the one or more stannous sources can be any suitable source of stannous ions. The one or more stannous ion sources can include, for example, stannous halides, organic stannous carboxylate salts, such as stannous formate, stannous acetate, stannous gluconate, stannous lactate, stannous tartrate, and stannous citrate. When the fluoride source is stannous fluoride, it can also function as a stannous source.

When employed, the one or more antimicrobial agents can include any orally acceptable antimicrobial agents. Examples include triclosan, 8-hydroxyquinoline, zinc ion, stannous ion, cupric compounds, phthalic acid and salts thereof, quaternary ammonium compounds, sanguinarine, salicylanilide, salicylic acid, thymol, eugenol, neomycin, kanamycin, clindamycin, amoxicillin, tetracycline, doxycycline, minocycline, metronidazole, chlorohexidine, and the like.

When employed, the one or more antioxidants can be any orally acceptable antioxidants. Examples include butylated hydroxy anisone, butylated hydroxy toluene, vitamin A, carotenoids, vitamin E, flavonoids, polyphenols, ascorbic acid or salts thereof, chlorophyll, melatonin, and the like.

When employed, the one or more saliva stimulants can be any orally acceptable saliva stimulants. Examples include citric acid, lactic acid, succinic acid, ascorbic acid, adipic acid, fumaric acid, and tartaric acid.

When employed, the one or more breath freshening agents can be any orally acceptable breath freshening agents. Examples include zinc salts such as zinc salts of gluconate, citrate, and chlorite, alpha-ionone, and the like.

When employed, the one or more antiplaque agents can be any orally acceptable antiplaque agents. Examples include stannous salts, salts of copper, magnesium or strontium, dimethicone copolyols, such as cetyl dimethicone copolyol, papain, glucamylase, glucose oxidase, urea, calcium lactate, calcium glycerophosphate, strontium polyacrylates, and the like. Further examples of antiplaque agents include biofilm inhibition agents, particularly those described in U.S. Pat. No. 8,968,709.

When employed, the one or more anti-inflammatory agents can be any orally acceptable anti-inflammatory agent. Examples include steroids such as flucinolone and hydrocortisone, non-steroidal anti-inflammatory drugs such as ketorolac, flubrbiprofen, ibuprogen, naproxen, indomethacin, diclofenac, etodolac, indomethacin, sulindac, tomlmetin, ketoprofen, fenoprofen, piroxicam, nabumetone, acetyl salicylic acid, salicylic acid, diflunisal, meclofenamate, mefenamic acid, oxyphenbutazone, phenylbutazone, and the like.

When employed, the one or more H₂ antagonists can be any orally acceptable H₂ antagonist. Examples include cimetidine, etinidine, ranitidine, tiotidine, lupitidine, denetidine, famotidine, roxatidine, pifatidine, lamtidine, zaltidine, nizatidine, mifentidine, ramixotidine, loxtidine, bisfentidine, sufotidine, ebrotidine, impromdine, and the like.

When employed, the one or more desensitizing agents can be any orally acceptable desensitizing agent. Examples include potassium citrate, potassium chloride, potassium tartrate potassium bicarbonate, potassium oxalate, potassium nitrate, strontium salts, arginine, acetyl salicylic acid or salt thereof, salicylic acid of salt thereof, codeine, acetaminophen, and the like.

When employed, the one or more nutrients can be any orally acceptable nutrients. Examples include vitamins, such as vitamins C, D, thiamine, riboflavin, folic acid, nicotinamide, niacin, pyridoxine, bioflavonoids, and the like, supplements, such as amino acids, lipotropics, fish oil, polyunsaturated fatty acids, eicosapentanoic acid, docosahexanic acid, coenzyme Q10, ubiquinone, minerals such as potassium, and the like.

When employed, the one or more proteins can include any orally acceptable protein. Examples include milk proteins, peroxide producing enzymes, amylase, papain, glucoamylase, glucose oxidase, and the like.

In use, the fluoride releasing composition can be applied to the oral cavity, typically in the form of a dental vanish. When applied as a dental varnish, the fluoride releasing composition is typically applied directly to one or more teeth. After application to the teeth, the fluoride releasing composition, which is often in the form of a varnish, sets. At body temperature, that is, 37° C., the fluoride releasing composition will typically set in no more than two minutes. Often, setting at 37° C. can be even faster, such as no more than 90 seconds, no more than one minute, or even no more than thirty seconds.

After setting, for example, on the teeth, the fluoride releasing composition the fluoride releasing composition releases fluoride, for example, in the oral cavity and at least partially into the teeth. The release of fluoride is typically measured in μg fluoride released per area of the set composition, for example, on the teeth, typically in cm². Thus, in many cases, at least 20 μg/cm² of the fluoride is released from the composition in no more than one hour after application. In other cases, at least 40 μg/cm² of the fluoride is released from the composition in no more than one hour after application. The release of fluoride is measured, for example, by the method described in the Examples section of this disclosure.

The fluoride releasing composition is typically maintained in contact with the teeth for a sufficient time to release a therapeutically effective amount of fluoride. In most cases, the fluoride releasing composition sets on the teeth and remains until physically removed, such as by brushing the teeth. Thus, the fluoride releasing composition can remain in contact with the teeth for at least 15 minutes, at least 30 minutes, at least 1 hour, at least 2 hours, at least 3 hours, at least 4 hours, at least 5 hours, at least 6 hours, at least 8 hours, at least 10 hours, at least 12 hours, at least 14 hours, at least 18 hours, at least 20 hours, at least 22 hours, or even at least 24 hours, before removal.

### List of Illustrative Embodiments

This list of embodiments is meant to aid in understanding particular aspects of the invention. It is not intended to be limiting.

1. A fluoride releasing composition acceptable for oral use, comprising:

50% to 70% by weight of a hydrogenated rosin acid, wherein greater than 17% and less than 85% of the hydrogenated rosin acid is in the form of neutralized hydrogenated rosin acid;

5% to 12% by weight of water;

12% to 20% by weight of ethanol; and

one or more fluoride sources comprising fluoride;

wherein the fluoride releasing composition has a consistency of at least 24 mm.

2. The fluoride releasing composition of embodiment 1, wherein the fluoride releasing composition is in the form of a dispersion.

3. The fluoride releasing composition of any of the preceding embodiments, wherein the hydrogenated rosin acid is present in at least 50%, at least 55%, at least 60%, or at least 65% by weight of the fluoride releasing composition.

4. The fluoride releasing composition of any of the preceding embodiments, wherein the hydrogenated acid rosin is present in no more than 65%, no more than 60%, or no more than 55% by weight of the fluoride releasing composition.

5. The fluoride releasing composition of any of the preceding embodiments, wherein the degree of neutralization of the hydrogenated rosin acid is greater than 17%, greater than 20%, greater than 25%, greater than 30%, greater than 35%, greater than 40%, greater than 45%, greater than 50%, greater than 55%, greater than 60%, greater than 65%, greater than 70%, greater than 75%, greater than 75%, or greater than 80% by weight of the fluoride releasing composition.

6. The fluoride releasing composition of any of the preceding embodiments, wherein the degree of neutralization is less than 85%, less than 80%, less than 75%, less than 70%, less than 65%, less than 60%, less than 55%, less than 50%, less than 45%, less than 40%, less than 35%, less than 30%, less than 25%, or less than 20% by weight of the fluoride releasing composition.

7. The fluoride releasing composition of any of the preceding embodiments, wherein the ethanol is present in at least 12%, at least 13%, at least 14%, at least 15%, at least 16%, at least 17%, at least 18%, or at least 19% by weight of the fluoride releasing composition.

8. The fluoride releasing composition of any of the preceding embodiments, wherein the ethanol is no more than 20%, no more than 19%, no more than 18%, no more than 17%, no more than 16%, no more than 15%, no more than 14%, or no more than 13% by weight of the fluoride releasing composition.

9. The fluoride releasing composition of any of the preceding claims, wherein at least a portion of the neutralized hydrogenated rosin acid is in the form of an alkali metal salt or an alkaline earth metal salt.

10. The fluoride releasing composition of embodiment 9, wherein the alkali or alkaline earth metal salt is potassium, sodium, magnesium, or calcium.

11. The fluoride releasing composition of embodiment 9, wherein the alkali or alkaline earth metal salt is potassium or sodium.

12. The fluoride releasing composition of any of the preceding embodiments, wherein the fluoride source is present in at least 0.1%, at least 0.5%, at least 1%, at least 2%, at least 3%, at least 4%, at least 5%, at least 6%, at least 7%, at least 8%, or at least 9% by weight of the fluoride releasing composition.

13. The fluoride releasing composition of any of the preceding embodiments, wherein the fluoride source is present in no more than 10%, no more than 9%, no more than 8%, no more than 7%, no more than 6%, no more than 5%, no more than 4%, no more than 3%, no more than 2%, no more than 1%, or no more than 0.5% by weight of the fluoride releasing composition.

14. The fluoride releasing composition of any of the preceding embodiments, wherein the fluoride source is present in 2.2% by weight of the fluoride releasing composition.

15. The fluoride releasing composition of any of embodiments 1-13, wherein the fluoride source is present in 2.6% by weight of the fluoride releasing composition.

16. The fluoride releasing composition of any of embodiments 1-13, wherein the fluoride source is present in 5% by weight the fluoride releasing composition.

17. The fluoride releasing composition of any of the preceding embodiments, wherein the composition is capable of setting after no more than 2 minutes at 37° C.

18. The fluoride releasing composition of any of the preceding embodiments, wherein the composition is capable of setting after no more than 90 seconds minutes at 37° C.

19. The fluoride releasing composition of any of the preceding embodiments, wherein the composition is capable of setting after no more than 60 seconds minutes at 37° C.

20. The fluoride releasing composition of any of the preceding embodiments, wherein the composition is capable of setting after no more than 30 seconds minutes at 37° C.

21. The fluoride releasing composition of any of the preceding claims, which is in the form of a dental varnish.

22. The fluoride releasing composition of any of the preceding embodiments, wherein at least one of the one or more fluoride sources is sodium fluoride or strontium fluoride.

23. The fluoride releasing composition of any of the preceding claims, further comprising a hydrogenated ester rosin.

24. The fluoride releasing composition of any of the preceding claims, further comprising at least one excipient selected from the group consisting of fillers, flavorings, and sweeteners.

25. The fluoride composition of any of the preceding embodiments, further comprising at least one filler is present and is selected from the group consisting of polyethylene glycol, polypropylene glycol, copolymers of polyethylene glycol and polypropylene glycol, silica, fumed silica, alumina, titania, zinc oxide, and glycerin.

26. The fluoride composition of embodiment 17, wherein the at least one filler is present in 0.5% to 15% by weight of the fluoride releasing composition.

27. The fluoride releasing composition of any of the preceding embodiments, wherein the at least one sweetener is present and is selected from the group consisting of xylitol, sorbitol, aspartame, saccharine, and sodium saccharine.

28. The fluoride releasing composition of embodiment 19, wherein the one or more sweeteners are present from 0.5% to 3% by weight of the fluoride releasing composition.

29. The fluoride releasing composition of any of the preceding embodiments, further comprising at least one additional active agent.

30. The fluoride releasing composition of any of the preceding embodiments, further comprising one or more additional active agents, wherein at least one of the one or more additional active agents are selected from whitening agents, anticalculus agents, stannous sources, antimicrobial agents, antioxidants, saliva stimulating agents, breath freshening agents, antiplaque agents, anti-inflammatory agents, H₂ antagonists, desensitizing agents, nutrients, and proteins.

31. The fluoride releasing composition of any of the preceding embodiments, wherein at least 20 μg/cm² of the fluoride is released from the composition in no more than one hour after application.

32. The fluoride releasing composition of any of the preceding embodiments, wherein at least 40 μg/cm² of the fluoride is released from the composition in no more than one hour after application.

33. The fluoride releasing composition of any of the preceding embodiments wherein at least 60% of the fluoride is released from the composition in no more than two hours after application.

34. The fluoride releasing composition of any of the previous embodiments, wherein the composition is free of hydrocarbon solvents.

35. The fluoride releasing composition of any of the previous embodiments, wherein the composition is free of alcohol solvents other than ethanol.

36. The fluoride releasing composition of any of the previous embodiments, wherein the consistency is measured by

(a) placing 0.15 g of liquid in the center of a first square glass plate having a side length of 10.16 cm (4 inches);

(b) suspending a second square glass plate having a side length of 10.16 cm (4 inches) and a mass of 110 g just above the sample; (c) releasing the second glass plate without applying any downward pressure; (d) allowing the second glass plate to remain in place for 120 seconds after release; (e) removing the second glass plate without disturbing the circle of sample formed on the first glass plate; and then (e) measuring the diameter of the circle of sample formed on the first glass plate in three locations at 120 degree intervals.

37. A method of applying fluoride to one or more teeth, the method comprising contacting the one or more teeth with a fluoride releasing composition of any of the previous embodiments.

38. The method of embodiment 34, further comprising setting the fluoride releasing composition on at least one of the one or more teeth.

39. The method of any of embodiments 34-36, wherein the step of setting the composition is complete within two minutes of contacting the fluoride releasing composition with the one or more teeth.

40. The method of any of embodiments 34-36, wherein the composition remains in contact with the one or more teeth for at least 15 minutes, at least 30 minutes, at least 1 hour, at least 2 hours, at least 3 hours, at least 4 hours, at least 5 hours, at least 6 hours, at least 8 hours, at least 10 hours, at least 12 hours, at least 14 hours, at least 18 hours, at least 20 hours, at least 22 hours, or at least 24 hours.

### Examples

The following examples illustrate particular aspects of the disclosure, but are not intended to be limiting to the scope of the claims. Unless otherwise specified, all parts and percentages are by weight and all water is deionized water. When the source of materials is not specified, the material is obtainable from Sigma-Aldrich (St. Louis, Mo., US). All commercial materials were used as received from the vendor.

### Materials

AEROSIL 200 Pharma is a fumed silica, which was obtained from Evonik, (Parsippany, N.J., US).

CALIPHARM D is dicalcium phosphate dihydrate, which was obtained from Multchler, (Harrington Park, N.J., US).

Ethanol (200 proof) was obtained from Columbus Chemical Industries, Inc. (Ashville, Ohio, US).

FORAL AX is a thermoplastic, hydrogenated acid rosin, which was obtained from Pinova, Inc. (Brunswick, Ga., US).

FORALYN 5020-F is a hydrogenated rosin ester, which was obtained from Eastman Chemical (Kingsport, Tenn., US).

Sodium fluoride (NaF) was obtained from (Sunlit Fluo & Chemical Company, Taiwan).

Sodium hydroxide (NaOH) was obtained from EMD Performance Materials (Damstadt, Germany).

Spearmint and bubble gum flavors were obtained from Foote & Jenks (Camden, N.J., USA.

Strontium fluoride (SrF₂) was obtained from Honeywell International Inc. (Morris Plains, N.J., US).

Sucralose was obtained from Sigma-Aldrich.

TCP-SLS is an abbreviation for sodium lauryl sulfate-treated tricalcium phosphate, which was prepared essentially as described in U.S. Pat. No. 9,023,373.

### Fluoride Release Testing

Approximately 50 mg of coating composition was painted evenly onto a 2.54 cm×2.54 cm (1 inch×1 inch) plastic slide (RINZYL plastic micro slide available from VWR, Radnor, Pa.). The coated slide was immersed in 25 mL of deionized water in a plastic test tube for 1 hour. After 1 hour, the slide was removed, rinsed and then immersed in a second 25 mL water in test tube. After 3 more hours (4 hours total), the process was repeated and the slide was immersed in third 25 mL aliquot of water. After 2 more hours (6 hours total) the process was again repeated and the slide was place in a fourth 25 mL aliquot of water where it remained for an additional 18 hours (24 hours total) before being removed. Each of the 25 mL aliquots of deionized water were then evaluated for fluoride concentration. 10 mL of the sample solutions from above preparation were mixed with 10 mL of TISAB II (Total Ionic Strength Adjustment Buffer II) to make the solution for fluoride concentration measurements. The fluoride concentrations were measured using a Cole Parmer fluoride ion meter equipped with a fluoride combination electrode which had been standardized using standard concentrations of fluoride buffered with TISAB II. Five replicate samples were run to obtain an average. The fluoride concentration was measured in parts per million and the fluoride release was reported as μF/cm² coating using the following equation.

μF/cm² coating=((Concentration of F in ppm)*(sample volume in mL))/(coating area in cm²)

Fluoride-releasing varnish compositions were prepared by thoroughly mixing the components according to the amounts indicated in Tables 1-2. Mixing was performed at a speed of 3000 rpm mixing speed on a DAC150 speedmixer from FlackTeck Inc, Landrum, S.C. Amounts of all materials in the tables are indicated in weight percent, (wt. %). The degree of neutralization was reached by adding a quantity of sodium hydroxide to neutralize the desired percentage of acid group on the hydrogenated rosin. The acid number of the hydrogenated rosin was 168 mg potassium hydroxide per gram of hydrogenated rosin; that value was provided by the manufacturer. The degree of neutralization (as a percent) can be calculated as follows:

acid number of rosin acid (g KOH/rosin acid)*g rosin acid used/g NaOH used*71.3

TABLE 1a
Varnish Compositions with Bubble Gum Flavor

| Component | EX1 | EX2 | EX3 | EX4 | EX5 | EX6 |
|---|---|---|---|---|---|---|
| NaOH | 3.56 | 3.58 | 3.36 | 3.38 | 2.655 | 2.685 |
| FORAL AX | 63.19 | 63.545 | 59.64 | 59.995 | 63.72 | 64.44 |
| DI water | 8.01 | 8.055 | 7.56 | 7.605 | 4.425 | 4.475 |
| Ethanol (200 proof) | 14.24 | 14.32 | 13.44 | 13.52 | 17.7 | 17.9 |
| NaF | 4 | 5 | 4 | 5 | 3 | 5 |
| SrF₂ | 1.5 | 0 | 1.5 | 0 | 3 | 0 |
| Sucralose | 0.5 | 0.5 | 0.5 | 0.5 | 0.5 | 0.5 |
| AEROSIL 200 | 2 | 2 | 2 | 2 | 2 | 2 |
| Pharma | | | | | | |
| Bubble gum flavor | 2 | 2 | 2 | 2 | 2 | 2 |
| FORALYN 5020-F | 0 | 0 | 5 | 5 | 0 | 0 |
| TCP-SLS | 0.5 | 0.5 | 0.5 | 0.5 | 0.5 | 0.5 |
| CALIPHARM D | 0.5 | 0.5 | 0.5 | 0.5 | 0.5 | 0.5 |
| Total | 100 | 100 | 100 | 100 | 100 | 100 |
| Total water^{¥} | 9.6 | 9.7 | 9.1 | 9.1 | 5.6 | 5.7 |
| Total ethanol^{¥} | 14.2 | 14.3 | 13.4 | 13.5 | 17.7 | 17.9 |
| Neutralization | 47.0 | 47.0 | 47.0 | 47.0 | 34.8 | 34.8 |
| degree % | | | | | | |
| Consistency/ | 43/0.9 | 42/0.9 | 42/0.7 | 43/1.0 | 45/0.8 | 47/0.9 |
| STDEV | | | | | | |
| Setting on slides | sticky | sticky | sticky | sticky | sticky | sticky |
| | coating | coating | coating | coating | coating | coating |

^{¥}Water from DI water and neutralization; bubble gum flavor did not include water (or ethanol).

TABLE 1b
Varnish Compositions with Bubble Gum Flavor

| Component | EX7 | EX8 | CE1 | CE2 |
|---|---|---|---|---|
| NaOH | 2.505 | 5.37 | 1.3425 | 6.265 |
| FORAL AX | 60.12 | 61.755 | 65.7825 | 60.86 |
| DI water | 4.175 | 8.95 | 8.95 | 8.95 |
| Ethanol (200 proof) | 16.7 | 13.425 | 13.425 | 13.425 |
| NaF | 3 | 5 | 5 | 5 |
| SrF₂ | 3 | 0 | 0 | 0 |
| Sucralose | 0.5 | 0.5 | 0.5 | 0.5 |
| AEROSIL 200 | 2 | 2 | 2 | 2 |
| Pharma | | | | |
| Bubble gum flavor | 2 | 2 | 2 | 2 |
| FORALYN 5020-F | 5 | 0 | 0 | 0 |
| TCP-SLS | 0.5 | 0.5 | 0.5 | 0.5 |
| CALIPHARM D | 0.5 | 0.5 | 0.5 | 0.5 |
| Total | 100 | 100 | 100 | 100 |
| Total water^{¥} | 5.3 | 11.4 | 9.6 | 11.8 |
| Total ethanol^{¥} | 16.7 | 13.4 | 13.4 | 13.4 |
| Neutralization | 34.8 | 72.6 | 17.0 | 85.9 |
| degree % | | | | |
| Consistency/ | 47/0.8 | 51/0.9 | 26/1.5 | 54/0.4 |
| STDEV | | | | |
| Setting on slides | Sticky | Sticky | NA | No setting |
| | coating | coating | | |

^{¥}Water from DI water and neutralization; bubble gum flavor did not include water (or ethanol).

TABLE 2
Varnish Compositions with Spearmint Flavor

| Component | EX9 | EX10 | EX11 | EX12 | EX13 | EX14 | EX15 |
|---|---|---|---|---|---|---|---|
| NaOH | 1.82 | 1.82 | 1.83 | 1.83 | 2.67 | 2.52 | 2.685 |
| FORAL AX | 67.34 | 67.34 | 67.71 | 67.71 | 62.3 | 58.8 | 62.65 |
| DI water | 8.19 | 4.55 | 8.235 | 4.575 | 8.9 | 8.4 | 8.95 |
| Ethanol (200 proof) | 13.65 | 17.29 | 13.725 | 17.385 | 15.13 | 14.28 | 15.215 |
| NaF | 4 | 4 | 5 | 5 | 4 | 4 | 5 |
| SrF₂ | 1.5 | 1.5 | 0 | 0 | 1.5 | 1.5 | 0 |
| Sucralose | 0.5 | 0.5 | 0.5 | 0.5 | 0.5 | 0.5 | 0.5 |
| AEROSIL 200 | 0 | 0 | 0 | 0 | 2 | 2 | 2 |
| Pharma | | | | | | | |
| Spearmint flavor | 2 | 2 | 2 | 2 | 2 | 2 | 2 |
| FORALYN 5020-F | 0 | 0 | 0 | 0 | 0 | 5 | 0 |
| TCP-SLS | 0.5 | 0.5 | 0.5 | 0.5 | 0.5 | 0.5 | 0.5 |
| CALIPHARM D | 0.5 | 0.5 | 0.5 | 0.5 | 0.5 | 0.5 | 0.5 |
| Total | 100 | 100 | 100 | 100 | 100 | 100 | 100 |
| Total water^{¥} | 9.1 | 5.4 | 9.1 | 5.5 | 10.2 | 9.6 | 10.2 |
| Total ethanol^{¥} | 15.2 | 18.8 | 15.2 | 18.9 | 16.6 | 15.8 | 15.2 |
| Neutralization | 22.6 | 22.6 | 22.6 | 22.6 | 35.8 | 35.8 | 35.8 |
| degree % | | | | | | | |
| Consistency/ | 40/1.2 | 50/1.2 | 40/0.3 | 52/0.9 | 32/0.7 | 34/1 | 34.7/1.0 |
| STDEV | | | | | | | |
| Setting on slides | Sticky | Sticky | Sticky | Sticky | Sticky | Sticky | Sticky |
| | coating | coating | coating | coating | coating | coating | coating |

^{¥}Spearmint flavor included water (3.8 wt.%) and ethanol (75 wt.%). The values for total water include DI water, water from neutralization, and water from spearmint flavor. The values for total ethanol include Ethanol (200 proof) and ethanol from spearmint flavor.

TABLE 3
Cumulative Fluoride release from Varnish Compositions (μg/cm²)

| Example | 1 hour | 4 hours | 6 hours | 24 hours |
|---|---|---|---|---|
| EX1 | 94 | 112 | 116 | 126 |
| EX2 | 137 | 152 | 155 | 163 |
| EX3 | 107 | 126 | 127 | 132 |
| EX4 | 138 | 156 | 159 | 165 |
| EX5 | 51 | 70 | 75 | 84 |
| EX6 | 79 | 105 | 120 | 147 |
| EX7 | 43 | 59 | 68 | 80 |
| EX9 | 36 | 58 | 68 | 90 |
| EX10 | 45 | 73 | 80 | 110 |
| EX11 | 43 | 71 | 88 | 116 |
| EX12 | 66 | 99 | 113 | 144 |
| EX13 | 81 | 100 | 108 | 119 |
| EX14 | 100 | 129 | 133 | 141 |
| EX15 | 81 | 90 | 96 | 107 |

1. A fluoride releasing composition acceptable for oral use, comprising:
50% to 70% by weight of a hydrogenated rosin acid, wherein greater than 17% and less than 85% of the hydrogenated rosin acid is in the form of neutralized hydrogenated rosin acid;
5% to 12% by weight of water;
12% to 20% by weight of ethanol; and
one or more fluoride sources comprising fluoride;
wherein the fluoride releasing composition has a consistency of at least 24 mm.

2. The fluoride releasing composition of claim 1, wherein the fluoride releasing composition is in the form of a dispersion.

3. The fluoride releasing composition of claim 1, wherein at least a portion of the neutralized hydrogenated rosin acid is in the form of an alkali metal salt or an alkaline earth metal salt.

4. The fluoride releasing composition of claim 1, wherein the composition is capable of setting after no more than 2 minutes at 37° C.

5. The fluoride releasing composition of claim 1, which is in the form of a dental varnish.

6. The fluoride releasing composition of claim 1, wherein at least one of the one or more fluoride sources is sodium fluoride or strontium fluoride.

7. The fluoride releasing composition of claim 1, further comprising a hydrogenated ester rosin.

8. The fluoride releasing composition of claim 1, further comprising at least one excipient selected from the group consisting of fillers, silica, flavorings, and, sweeteners.

9. The fluoride releasing composition of claim 1, wherein at least −20 microgram/square centimeter of the fluoride is released from the composition in no more than one hour after application.

10. The fluoride releasing composition of claim 1, wherein at least 40 microgram/square centimeter of the fluoride is released from the composition in no more than one hour after application.

11. A method of applying fluoride to one or more teeth, the method comprising contacting the one or more teeth with a fluoride releasing composition of any of the previous claims.

12. The method of claim 11, further comprising setting the composition on at least one of the one or more teeth.

13. The method of claim 11, wherein the composition sets within 2 minutes of contacting the one or more teeth.

14. The method of any of claim 11, wherein the composition remains in contact with the one or more teeth for at least 15 minutes.

15. The method of any of claim 11, wherein the composition remains in contact with the one or more teeth for at least one hour.
